(19) United States
(12) Patent Application Publication
(10) Pub. No.: US 2014/0148803 A1
(43) Pub. Date: May 29, 2014

(54) EXTERNAL ACTUATOR FOR AN ELECTROSURGICAL INSTRUMENT

(71) Applicant: COVIDIEN LP, Mansfield, MA (US)

(72) Inventor: CODY C. TAYLOR, BOULDER, CO (US)

(73) Assignee: COVIDIEN LP, Mansfield, MA (US)

(21) Appl. No.: 14/068,739

(22) Filed: Oct. 31, 2013

Related U.S. Application Data
(60) Provisional application No. 61/730,597, filed on Nov. 28, 2012.

(51) Int. Cl. A61B 18/14; A61B 18/12

(52) U.S. Cl. CPC A61B 18/14; A61B 18/1206
USPC 606/34; 606/41; 606/49

(57) ABSTRACT

An electrosurgical instrument is provided having a treatment portion attached at a distal end thereof and a handle for actuating the treatment portion. The instrument includes at least one switch, each configured to close an associated open circuit upon activation thereof for controlling at least one respective function or parameter associated with the treatment portion. The instrument is provided with a cap configured to mechanically engage the housing and having an inner surface including a corresponding number of mechanical interfaces configured to align with each switch, such that an activation force against an outer surface of the cap and relative to the housing closes the open circuit associated with a corresponding switch.

### CROSS REFERENCE TO RELATED APPLICATION

The present application claims the benefit of and priority to U.S. Provisional Application Ser. No. 61/730,597, filed on Nov. 28, 2012, the entire contents of which are incorporated herein by reference.

### BACKGROUND

1. Technical Field

The present disclosure relates to handheld electrosurgical instruments and, more particularly, to a user controllable actuator externally provided on the housing of the electrosurgical instrument.

2. Background of Related Art

Handheld electrosurgical instruments, e.g., forceps, are used for treating tissue, including applying energy to, clamping, coagulating, cauterizing, sealing, and/or stapling tissue. A surgeon holds the electrosurgical instrument by a handpiece assembly during an electrosurgical procedure. The electrosurgical instrument is provided with several actuators. A first actuator controls application of the electrosurgical energy, e.g., via a pair of jaw members. A second actuator, such as a lever, may control opening and closing of the jaw members relative to one another for clamping and releasing tissue. A third actuator, such as a trigger, may control deployment of a knife for severing tissue.

During the procedure, the surgeon operates the first actuator in order to apply energy to selected tissue at the surgical site while the surgeon manipulates the electrosurgical instrument and/or tissue. The surgeon needs the ability to comfortably reach and operate the all three actuators during the electrosurgical procedure.

Internal space defined within the housing may be crowded with components. This limits internal space available for interfacing the first actuator with internal components. For example, in a portable electrosurgical instrument, a battery may be configured to removably couple or “latch” to the housing of the portable surgical instrument, thus forming part of the handpiece assembly. This configuration eliminates the need for cumbersome power cords that couple to an external energy source. However, since the battery consumes a high percentage of the available internal space of the handpiece assembly, arranging other mechanisms and electronic components in the housing becomes a design challenge for manufacturers.

### SUMMARY

One aspect of the present disclosure relates to an electrosurgical instrument having a housing having a treatment portion attached at a distal end thereof and a handle for actuating the treatment portion. The instrument includes a plurality of switches, each configured to close an associated open circuit upon activation thereof for controlling respective functions or parameters associated with the treatment portion. The instrument is provided with a cap configured to mechanically engage the housing and having an inner surface including a corresponding number of mechanical interfaces configured to align with each switch, such that an activation force against an outer surface of the cap and relative to the housing closes the open circuit associated with a corresponding switch.

The functions controlled by the switches are selected from the group consisting of coagulation, homeostasis, RF sealing, ultrasonic sealing, blending, fulguration, coagulation, cauterization, cutting, and stapling tissue. The parameters controlled by the switches are selected from the group consisting of treatment mode, energy signal amplitude, energy signal frequency, energy signal waveform shape, energy signal duty cycle, and energy signal root mean square. The electrical signals are selected from the group of energy signals consisting of electrical current, electrical voltage, electrical power, and ultrasound signal.

In accordance with one aspect of the present disclosure, the cap may be ergonomically configured to wrap around the housing. The switches are disposed on a printed circuit board that is coupled to an external surface of the housing.

The housing has a longitudinal axis. When a first switch is depressed in a direction along the longitudinal axis of the housing by a corresponding mechanical interface, a first function or parameter associated with the treatment portion is controlled. When a second switch is depressed in a direction along the longitudinal axis of the housing by a corresponding mechanical interface, a second function or parameter associated with the treatment portion is controlled.

The switches are configured as dome switches each having a first and second terminal and a cover with a conductive inner surface. The open circuit associated with a corresponding switch is closed when the corresponding mechanical interface applies a second force to an outer surface of the cover, causing the cover to move from an initial first position in which the inner surface of the cover does not electrically conduct between the first and second terminals, to a second position in which the inner surface does electrically conduct between the first and second terminals.

In another aspect of the present disclosure the instrument may further include at least one tangible processing device and a module including a plurality of programmable instructions executable by the processing device for receiving at least one control signal indicative of actuation of two or more switches, and for controlling the respective functions or parameters based on which switch is actuated, or based on timing between successive actuations, as indicated by the received at least one control signal.

The module may operate in a first and second mode. When operating in the first mode, upon actuation of one of the switches the module controls a first function or parameter. When operating in the second mode, upon actuation of the switch, the module controls a second function or parameter.

The instrument may include an electro surgical generator and a knife driver configured to operate a knife for performing a cutting operation. The first function may include controlling the electrosurgical generator to perform a sealing operation that includes generating electrosurgical energy for performing a seal operation to seal tissue, and the second function may control the knife driver to perform a cutting operation to sever tissue.

The module may begin operating in an initial resting mode, and transition from operating in the initial resting mode to operating in the first mode upon actuation of the at least one switch in a first predetermined fashion. The module may transition from operating in the first mode to operating in the second mode upon completion of a successful seal operation. The module may control terminating an active sealing operation. Upon actuation of the at least one switch in a second predetermined fashion different from the first predetermined fashion, the module may transition from operating in the first or second mode to operating in the initial resting mode.

Yet another aspect includes an electrosurgical instrument having a housing having a treatment portion attached at a distal end thereof and a handle for actuating the treatment portion. At least one switch is provided, each configured to close an associated open circuit upon activation thereof for controlling at least one respective function or parameter associated with the treatment portion. The at least one switch is disposed on a printed circuit board that is coupled to an external surface of the housing.

The at least one switch may be configured as a dome switch having a first and second terminal and a cover with a conductive inner surface. The open circuit associated with a corresponding switch is closed when a force is applied to an outer surface of the cover, causing the cover to move from an initial first position in which the inner surface of the cover does not electrically conduct between the first and second terminals, to a second position in which the inner surface does electrically conduct between the first and second terminals.

The instrument may include a single cap having an inner and outer surface configured to couple to the housing so that the circuit board is disposed in between the cap and the housing with its outer surface exposed and its inner surface having a number of mechanical interfaces configured to align with each switch. Application of an activation force against an outer surface of the cap and relative to the housing closes the open circuit associated with a corresponding switch of the at least one switch.

The electrosurgical instrument may further include at least one tangible processing device and at least one software module including a plurality of programmable instructions executable by the at least one processing device for receiving at least one control signal indicative of actuation of two or more switches and controlling the at least one respective function or parameter based on which switch is actuated, or based on timing between successive actuations, as indicated by the received at least one control signal.

Still another aspect of the present disclosure includes an electrosurgical instrument having a housing having a treatment portion attached at a distal end thereof and a handle for actuating the treatment portion. An electromechanical interface is provided for coupling to an electrosurgical energy generator that generates electrosurgical energy which is provided to the treatment portion. At least one switch is disposed on a printed circuit board that is coupled to an external surface of the housing and configured to close an associated open circuit upon activation thereof for controlling operation of the electrosurgical generator.

The electrosurgical instrument may further include a single cap configured to mechanically engage the housing so that the circuit board is disposed in between the cap and the housing so that the cap's outer surface is exposed and its inner surface faces the plurality of switches. Application of a first force against the outer surface of the cap and relative to the housing closes the open circuit associated with a corresponding switch.

In other aspects, closing the associated open circuit further controls at least one additional function or parameter associated with the treatment portion. The electrosurgical instrument further includes at least one tangible processing device and at least one software module including a plurality of programmable instructions executable by the at least one processing device. The software module receives at least one control signal indicative of actuation of one or more switches and further controls the electrosurgical generator and the at least one additional function or parameter based on which switch is actuated, or based on timing between successive actuations as indicated by the received at least one control signal.

### BRIEF DESCRIPTION OF THE DRAWINGS

Various embodiments of the present disclosure are described herein with reference to the drawings, wherein:

FIG. 1 is a side view of an example electrosurgical instrument in accordance with an embodiment of the present disclosure;

FIG. 2 is a rear, perspective view of the electrosurgical instrument shown in FIG. 1;

FIG. 3 is a rear view of the electrosurgical instrument shown in FIG. 1

FIG. 4 is a rear, perspective view of the upper portion of the electrosurgical instrument shown in FIG. 1, with an actuator shown in exploded form;

FIG. 5A is a top view of an exemplary flex circuit of the activation switch shown in FIG. 4;

FIG. 5B is a bottom view of the flex circuit shown in FIG. 5A;

FIG. 6 is a side, partial cross-sectional view of a body of the electrosurgical instrument shown in FIGS. 1-3, wherein a portion of the housing has been removed to show the internal and external placement of the flex circuit shown in FIGS. 5A and 5B;

FIG. 7A is a top view of an exemplary switch contact segment of the flex circuit shown in FIGS. 5A and 5B;

FIG. 7B is a schematic circuit diagram of exemplary circuitry associated with a plurality of control switches provide on the switch contact segment shown in FIG. 7A;

FIG. 8A is a top view of an exemplary assembled control switch in accordance with the present disclosure;

FIG. 8B is a side view of the control switch shown in FIG. 8A disposed in an initial position;

FIG. 8C is a side view of the control switch shown in FIG. 8A disposed in a depressed position;

FIG. 9A is a perspective view of an outer surface of an exemplary wraparound cap of the of the electrosurgical instrument in accordance with an embodiment of the present disclosure;

FIG. 9B is a perspective view of an inner surface of the wraparound cap shown in FIG. 9A;

FIG. 9C. is a rear view of the inner surface of the wraparound cap shown in FIG. 9B;

FIG. 10A is an exemplary block diagram of a plurality of control switches, a processing device and controlled devices in accordance with an embodiment of the present disclosure;

FIG. 10B is an exemplary block diagram of an alternative embodiment of the coupling between the plurality of switches and the processing device shown in FIG. 10A; and

FIG. 11 is a flowchart showing an exemplary method executed by the processing device shown in FIG. 10A.

### DETAILED DESCRIPTION

Embodiments of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

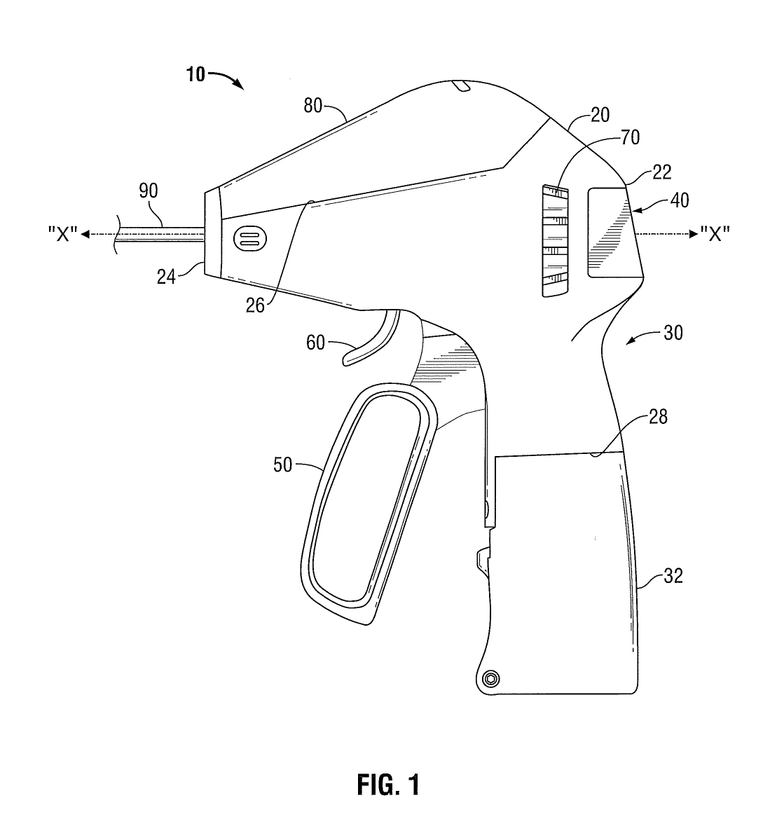

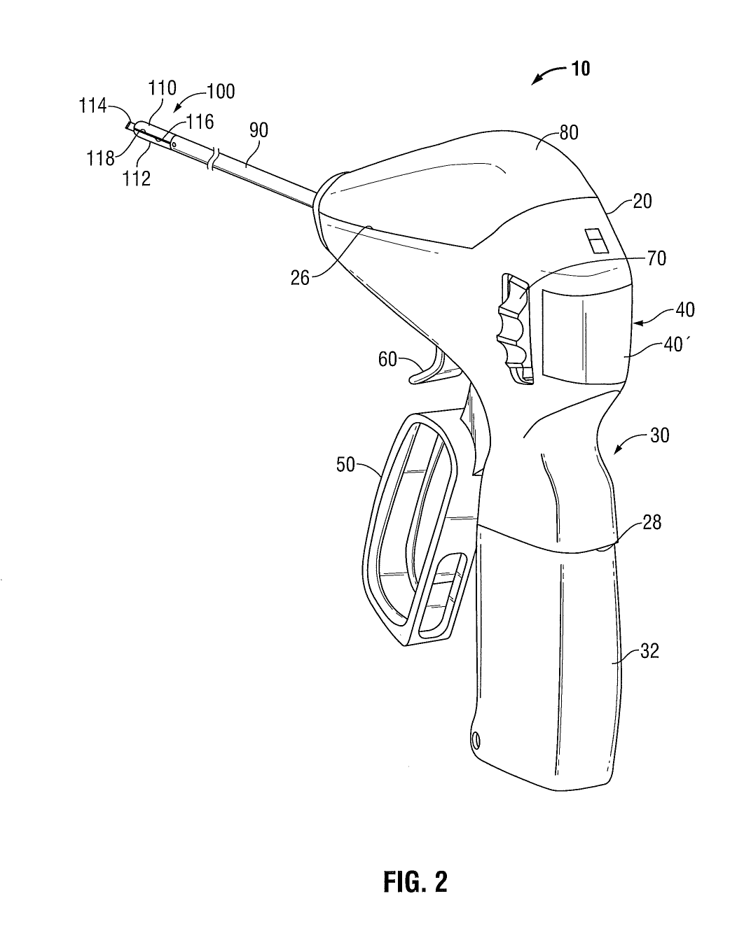

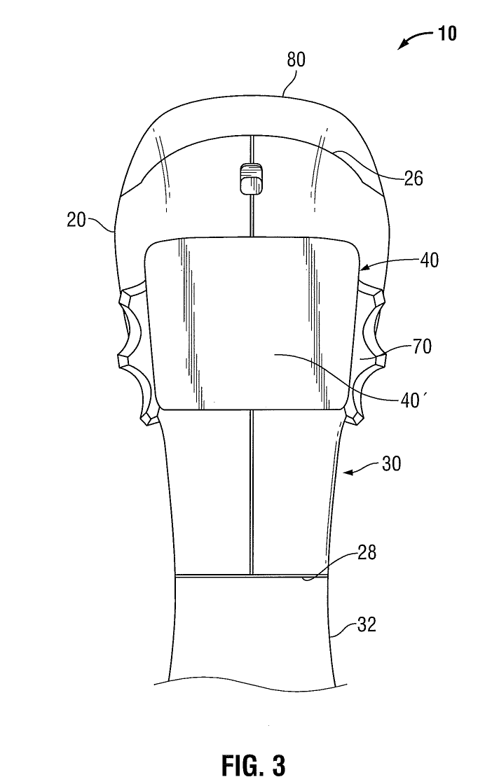

Turning now to FIGS. 1-3, electrosurgical instrument 10 is an example of an instrument for use in accordance with the present disclosure. Instrument 10 includes an outer-most housing 20, a handpiece assembly 30, an activation switch 40, a lever 50, a trigger 60, a rotation actuator 70, a generator 80, and a shaft 90 having an axis X-X defined therethrough. A treatment portion, here shown as exemplary end effector assembly 100, may be mechanically engaged to a distal end of shaft 90.

The treatment portion may include one or more stationary or moveable mechanisms for effecting a surgical task, such as applying energy to tissue, stapling, scraping, grasping, clamping, cauterizing, coagulating, desiccating, and/or cutting. For illustrative purposes the instrument 10 is described as a forceps having a pair of opposing jaw members 110, 112. Other varieties of surgical instruments are envisioned that deliver energy for operating the instrument and/or for applying energy to tissue. Both jaw members 110, 112 may be moveable with respect to the other, such as for grasping and manipulating tissue. Further, one or both of the jaw members 110, 112 may include energy application surfaces 116, 118, respectively, that may be disposed in opposed relation relative to one another. Energy, e.g., RF energy or ultrasound energy, may be supplied to one or both application surfaces 116, 118 of the jaw members 110, 112, such as to seal or otherwise treat tissue grasped therebetween. A knife 114 may be provided that is selectively advanceable between the jaw members 110, 112, e.g., to sever tissue disposed therebetween.

Instrument 10 includes several actuators that are selectively actuatable by the surgeon for controlling respective operations of instrument 10. For example, activator 40 controls delivery of energy in response to actuation, lever 50 controls operation of the end effector assembly 100, e.g., for opening and closing movement of the jaw members 110, 112 relative to one another for clamping and releasing tissue, trigger 60 controls deployment of the knife 114, and rotation actuator 70 controls rotation of shaft 12 about longitudinal axis X-X, e.g., for rotating the end effector assembly 100 and/or the knife 114.

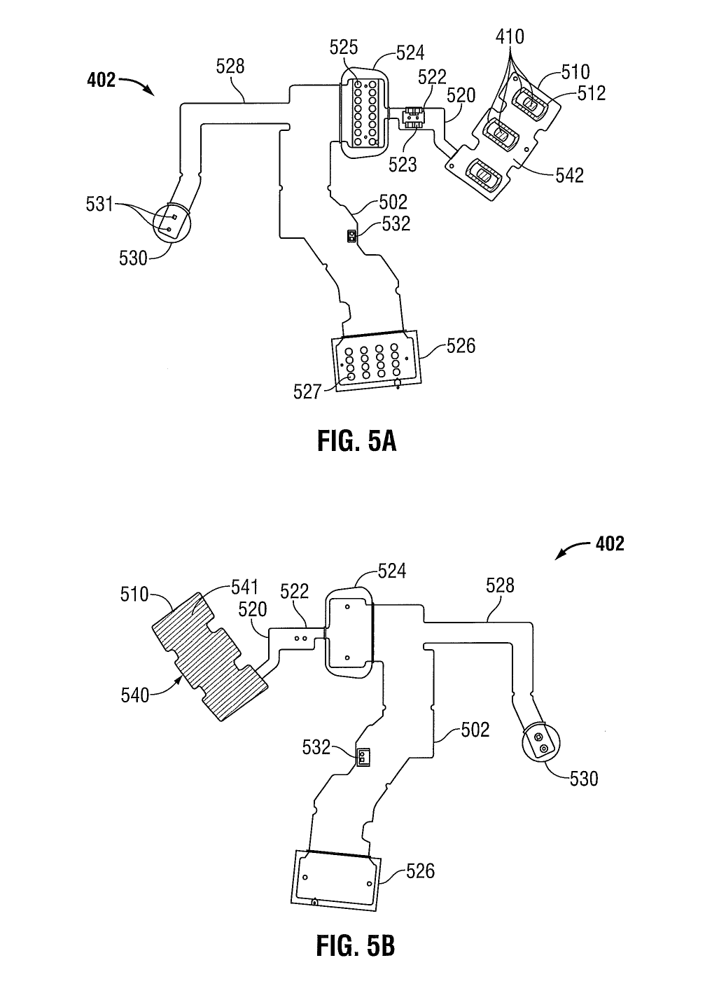

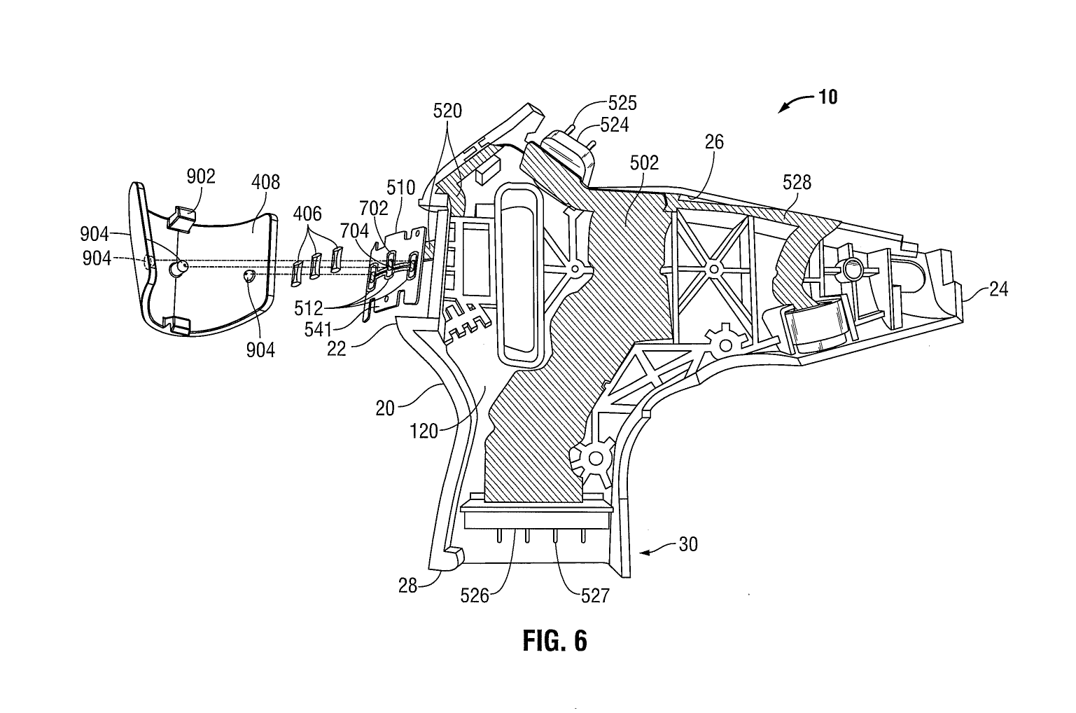

Housing 20 defines an internal space 120 (see FIG. 6) that extends between proximal and distal ends 22 and 24, respectively, of housing 20, upper wall 26, and lower wall 28. Upper wall 26 includes an electrical/mechanical interface 524 (See FIGS. 5-6) that interfaces with internally disposed generator 80. Lower wall 28 of handpiece assembly 30 includes an electrical/mechanical interface 526 (See FIGS. 5-6) for interfacing with a battery 32 or other energy source. The internal space 120 defined by housing 20 is thus limited since it is bounded by the geometrical limitations (size and shape) of the generator 80 and the battery 32.

Typically, the internal space 120 defined by the housing 20 holds electrical and mechanical components that interface with the respective actuators and the components that they control. With respect to the presently disclosed instrument 10, a portion of the mechanical/electrical interface 510, 520 (see FIG. 5A) associated with activator 40 is disposed external to housing 20. This is advantageous when the internal space 120 defined by housing 20 is limited, such as when instrument 10 is portable and provides for attachment of the battery 32 and generator 80 to the housing 20.

Battery 32 is configured to electrically couple to generator 80 for powering generator 80. Generator 80, in turn, supplies the desired energy to the energy application surfaces of the jaw members 110, 112. Generator 80 may include an output stage (not shown) that modulates the energy output by adjusting waveform parameters, e.g., waveform shape, pulse width, duty cycle, crest factor, and/or repetition rate. Alternatively, forceps 10 may be configured to couple to an external power source (not shown) and/or external generator (not shown), e.g., via an electrosurgical cable (not shown).

Both battery 32 and generator 80 operably couple to housing 20 and may be selectively removable therefrom. Generator 80 is powered by electrical energy supplied by battery 32 to generate electrosurgical energy, e.g., ultrasonic energy or RF energy for delivery to a patient.

Handpiece assembly 30 and battery 32 may be configured when assembled so that the surgeon holds instrument 10 by grasping handpiece assembly 30 together with battery 32. Handpiece assembly 30, battery 32, and the actuators may be ergonomically configured such that, when the surgeon holds instrument 10 the surgeon is able to operate activator 40, lever 50, trigger 60, and/or rotation actuator 70 with a single hand.

Activator 40 may include an ergonomically-dimensioned wrap-around switch 40′, the shape of which conforms to the outer shape of housing 20 when assembled. The wrap-around configuration envelopes the proximal end of housing 20 on both sides of longitudinal axis X-X, permitting the surgeon to activate switch 40′ from a variety of different orientations, e.g., multi-orientation activation. In one embodiment, the wrap-around switch 40′ symmetrically wraps around both sides of housing 20. In this way, switch 40′ can be comfortably operated by a right or left-handed surgeon.

Optionally, or alternatively, activation of switch 40′ from the different orientations may result in different functionality, including selecting an operational mode from a variety of operational modes or controlling one or more functions or parameters associated with the treatment portion. Functions that may be controlled include coagulation, homeostasis, RF sealing, ultrasonic sealing, blending, fulguration coagulation, cauterization, cutting, and stapling tissue. Parameters that may be controlled include amplitude, frequency, waveform shape, duty cycle, and root mean square of an energy signal. The energy signal controlled may include electrical current, electrical voltage, electrical power, and ultrasound signals.

For example, in a default mode (which may be designed as the only mode) activator 40 is configured as a toggle switch, wherein depression of activator 40 results in delivery of energy to the intended destination, which in the current example is the end effector assembly 100. Release of the activator 40 terminates delivery of the energy. The energy (e.g., RF or ultrasound) provided to the end effector assembly 100 is applied to a patient for surgical and/or therapeutic purposes.

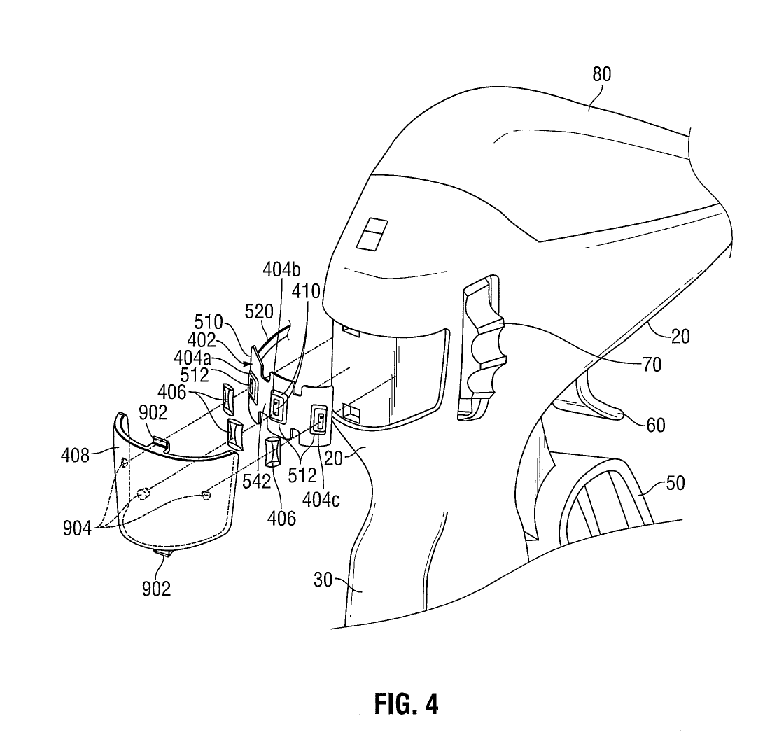

With reference to FIGS. 4-6, activator 40 includes a flexible circuit board 402, a plurality of control switches 404a-404c, and a wraparound cap 408. Flexible circuit board 402 includes a flexible, nonconductive substrate provided with a plurality of electrically conductive conduits 410 (e.g., formed of conductive ink). While three control switches 404a-404c have been disclosed herein, other quantities and types of control switches 404a-404c are contemplated.

Switch contact segment 510 of flexible circuit board 402 is positioned on an external surface of housing 20 or exposed through housing 20, including generator connector 524 which couples with a mating connector (not shown) provided on generator 80. The exposed outer surface of connector 524 is provided with a plurality of pins 525 configured to mate with the connector (not shown) provided on generator 80. A portion 522 of second arm 520 provides a mounting pattern for a through-hole pcb mounted switch (not shown). The exposed outer surface or switch actuator of this pcb mounted switch is accessible by the user of the device.

Each control switch 404a-404c includes a switch contact 512 and a contact cover 406. (See FIG. 4.) In one embodiment, the control switch 404a-404c is a snap dome switch and the contact cover 406 is a snap dome contact cover that deflects and inverts to establish electrical continuity when depressed on an outer surface there at, and returns or snaps to a pre-depressed configuration and original shape when the force is removed.

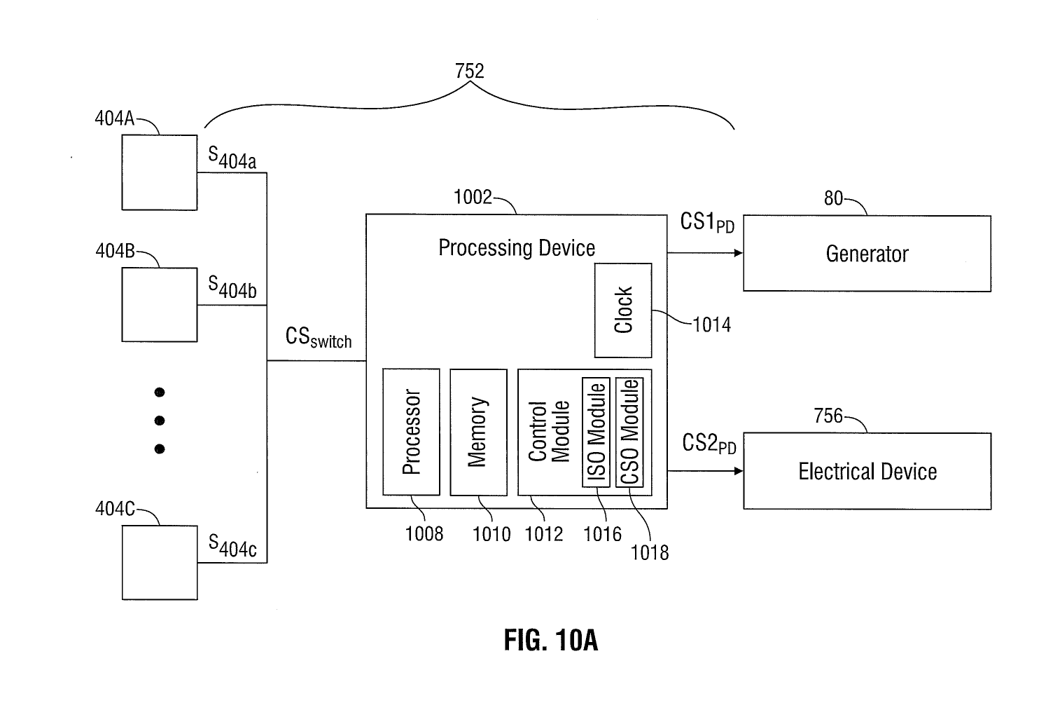

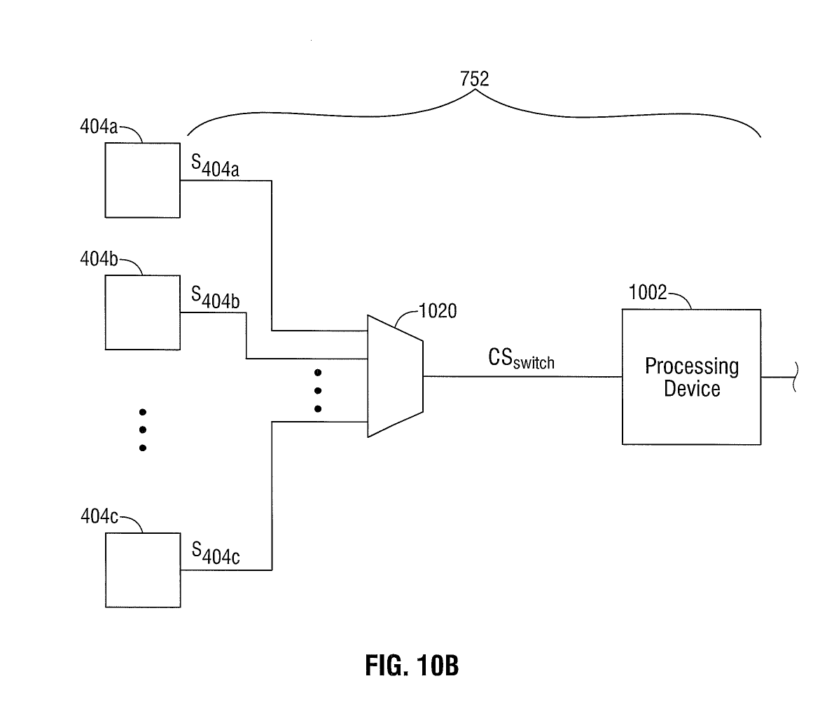

A plurality of switch contacts 512 are provided on the outer surface of switch contact segment 510. Each switch contact 512 interacts with an associated contact cover 406. When a contact cover 406 is depressed an associated electrical circuit is closed that actuates transmission of energy to the end effector assembly 100. More specifically, with additional reference to FIGS. 10A-10B, when the circuit associated with a control switch 404a-404c is closed, a control signal CS_{switch} is generated which is provided to a processing device 1002, located in the generator assembly that acts upon the signal. Processing device 1002 may generate additional control signals responsive to actuation of switches 404a-404c for controlling additional devices of the instrument 10, as will be described in greater detail below.

With returned reference to FIGS. 4-6, flexible circuit board 402 further includes a first arm 502 that extends inside the housing 20, (e.g., within the internal space 120 defined by housing 20) from generator connector 524 to a battery connector 526. Battery connector 526 is provided with a plurality of pins 527 configured to mate with corresponding connectors (not shown) provided on battery 32.

Flexible circuit board 402 further includes a second arm 520 that extends from generator connector 524 to switch contact segment 510. Second arm 520 extends so that switch contact segment 510 may be positioned on the external proximal face of housing 20. A hermetic seal (not shown) is provided on housing 20 to reduce the infiltration of foreign substances such as particles or fluids present at the surgical site.

A back surface 540 of switch contact segment 510 may include an adhesive layer 541 that attaches to the external surface of housing 20. Adhesive layer 541 secures switch contact segment 510 to the external surface of housing 20, with a top surface 542 thereof and switch contacts 512 exposed to the external environment of instrument 10.

Flexible circuit board 402 further includes a third arm 528 that extends inside housing 20 from arm 502 near its juncture with switch contact segment 525 to the speaker component 530 that provides output power to the jaws 110, 112. When assembled, speaker connector 530 is seated inside of internal housing 20. An additional connector 532 is provided on arm 502 inside housing 20 for coupling with a mating connector (not shown) of an internal component.

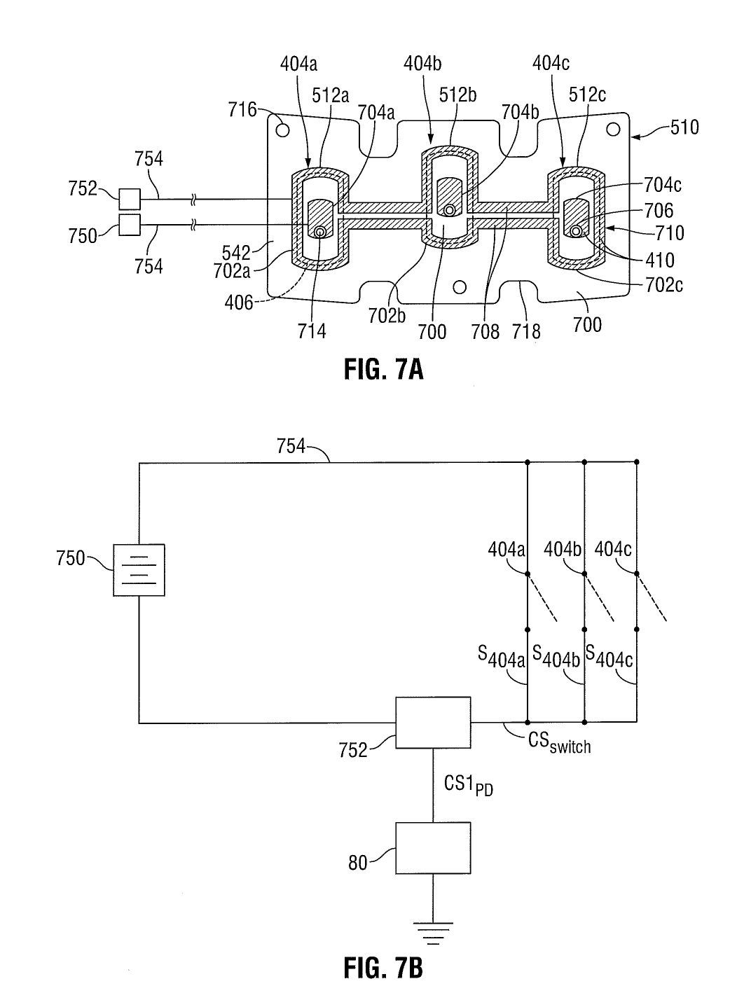

Turning attention to FIG. 7A, exemplary switch contact segment 510 includes a nonconductive substrate 700 formed of a flexible electrically insulating material, e.g., polyester, having layers of non-conductive (dielectric) ink and conductive ink defined thereon (e.g., screen printed) which define one or more electrical circuit patterns 710.

Electrical circuit pattern 710 includes outer terminals 702a-702c, inner terminals 704a-704c, and bridge terminal 708 that electrically couples 702a-702c, as shown in FIG. 7A. Alternatively, bridge 708 may be fully or partially omitted so that two or more of the electrical circuit patterns 710 associated with each switch contact 512 are electrically isolated from one another, as shown in FIG. 5A. Contact covers 406 (shown phantomed) associated with each contact switch 404a-404c are assembled in place over the respective switch contacts 512. Contact covers 406 are depressed (e.g., by applying a force in direction F1) to form an electrical connection between the outer and inner terminals 702 and 704 for forming an electrical circuit. Inner terminals 704a-704c are electrically coupled an electrical circuit or device 750, hereinafter referred to as electrical device 750. Outer terminals 702a-702c are electrically coupled an electrical circuit or device 752, hereinafter referred to as electrical device 752.

Outer apertures 716 are available for inserting a fastener, such as a stud, pin, or peg that aligns and fastens switch contact segment 510 to the external surface of housing 20 to supplement adhesion thereof. Notches 718 may be provided to accommodate the shape of housing 20. While a continuous oval-shaped outer terminal 702 and an inner circular terminal 704 has been disclosed herein, other geometric configurations for one or both the outer and inner terminals 702 and 704 are envisioned, e.g., circular, arcuate, semicircular, and the like.

FIG. 7B shows a schematic circuit diagram including control switches 404a-404c. The inner terminal 704 of each control switch 404a-404c is electrically coupled to first electrical device 750 by an electrical lead 714 that extends through substrate 700 to the first electrical device 750. First electrical device 750 may include, for example, a power, current, or voltage source (herein referred to as a current source), such as battery 32. The coupling of inner terminal 704 to the first electrical device 750 initially forms an open circuit.

The outer terminal 702 of each of control switches 404a-404c is electrically coupled by an electrical lead 754 shown in FIGS. 7A and 7B that extends through substrate 700 to a second electrical device 752 shown in FIG. 7A that is disposed external to contact segment 510. The second electrical device 752 may be a load, such as a controller (e.g., processing device 1002 shown in FIG. 10) or alternatively at least one electrical device 756. The electrical devices 756 may include, for example, audio or visual indicator device(s), and/or an electrical drive device (e.g., for driving deployment of the knife 114, opening and closing of the jaw members of the end effector assembly 100, or stapling with a surgical staple).

In another embodiment, external terminal 702 and inner terminal 704 can exchange roles; e.g., their functions are exchanged (e.g., outer terminal 702 is coupled to the second electrical device 752 and the first terminal 704 is coupled to the first electrical device 750).

Initially, when any of control switches 404a-404c are in an open state (shown by dotted lines) the switch contact's outer terminal 702 is electrically isolated from the associated inner terminal 704 while at least one of the outer terminal 702 or inner terminal 704 is coupled to first electrical device 750, thus forming an open circuit. When any of control switches 404a-404c is actuated and assumes a closed state, e.g., by actuating activator 40, a corresponding outer terminal thereof 702a-702c is electrically coupled to a corresponding inner terminal 704a-704b, and the actuated control switch 404a-404c transmits a signal S₄₀₄ₐ, S_{404b}, and S_{404c}, respectively. The coupling of the outer terminals 702a-702c and inner terminals 704a-704c of any of the control switches 404a-404c forms a closed electrical circuit 754 that includes at least first electrical device 750, the actuated control switch 404a, b or 404c, and second electrical device 752.

FIG. 7B shows signals S₄₀₄ₐ, S_{404b}, and S_{404c} provided directly to second electrical device 752. As described below in connection with FIGS. 10A and 10B, second electrical device 752 may include circuitry to combine, process, or modify signals S₄₀₄ₐ, S_{404b}, and S_{404c} generated by control switches 404a-404c, respectively, before the resulting signal CS_{switch} is provided to processing device 1002.

Thus, each switch contact 512a-512c is configured so that when corresponding inner terminals 704a-704c and outer terminals 702a-702c are electrically coupled, an initially open electrical circuit is closed. In one embodiment, coupling corresponding outer terminals 702a-702b and inner terminals 704a-704c and the associated closing of the electrical circuit 754 results in transmission of an activation signal to the generator 80 for activating application of electrosurgical energy to a patient via the end effector assembly 100. The activation signal may be transmitted via the closed circuit 754 directly to the generator 80, or alternatively, the activation signal may be transmitted to intervening circuitry (analog and/or digital) (not shown) that modifies and/or processes the activation signal before it is transmitted to the generator 80. The intervening circuitry may include, for example, an amplifier, filter, microprocessor, or microcontroller.

Release of any of the control switches 404a-404c opens that control switch 404a-404c and terminates transmission of the corresponding signal S₄₀₄ₐ-S_{404c}. In accordance with the exemplary configuration shown in FIG. 7B, when all of the control switches 404a-404c are opened, circuit 754 is opened and second electrical device 752 controls generator 80 so that activation of generator 80 is terminated, e.g., delivery of electrosurgical energy to the patient is prevented. Alternatively or additionally, second electrical device 752 may generate a control signal, such as CS2_{PD} (see FIG. 10A) for controlling another device, such as electrical device 756.

The circuit shown in FIG. 7B is exemplary and other configurations of coupling control switches 404a-404c to the first and second electrical devices 750, 752 are envisioned. Multiple first electrical devices 750 and second electrical devices 752 may be provided. Inner terminals 704a-704c of two or more of control switches 404a-404c may be connected to the same first electrical device 750, or each inner terminal 704a-704c may be connected to a different first electrical device 750. Likewise, outer terminals 702a-702c of two or more of control switches 404a-404c may be connected to a same second electrical device 752, or each outer terminal 702a-702c may be connected to respective different second electrical devices 752.

While control switches 404a-404c are shown connected in parallel (e.g., via bridge 708) it is envisioned that two or more of control switches 404a-404c may be connected in series or to independent circuits (e.g., by eliminating at least a portion of bridge 708). Circuitry may be provided for managing a situation in which two or more control switches 404a-404c are actuated simultaneously and appropriately control device 756 according to design choice.

Since each control switch 512a-512c may be coupled to a different second electrical device, each control switch 404a-404c may control operation of a different function. Additionally, as described further below in connection with FIG. 10, each consecutive actuation of a control switch 404a-404c, or actuation of each particular control switch 404a-404c may control different functions. Examples of functions that actuation of a control switch 404a-404c may control are entering a different operational state, controlling the characteristics of a signal, such as its amplitude, duty cycle, frequency, etc., and selecting a power curve that governs control of at least one character of the signal. Additionally, signals S₄₀₄ₐ, S_{404b}, and S_{404c}, and/or CS_{switch} may be used in a variety of ways for controlling device 756, such as wherein the rising or falling edges of one or more of the signals are used to control operation of the electrical device 756.

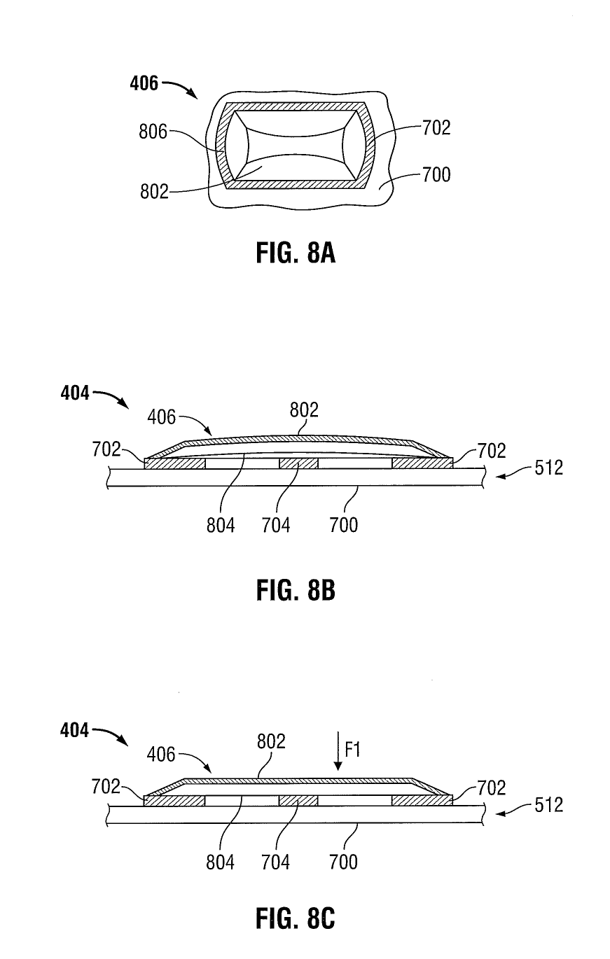

With additional reference to FIGS. 8A-8C, an exemplary snap dome contact cover 406 is assembled with each of the switch contacts 512. Contact cover 406 is a dome shaped switch cover formed of a conductive, resilient material, such as a suitable metal. The top surface of contact cover 406 is provided with a dielectric layer 802, such as an elastomeric/flexible insulating or non-conductive material. Dielectric layer 802 may coat the top surface of dome contact cover 406 and further extend outwardly to secure contact cover 406 to substrate 700 and seal the area of switch contact 512 surrounded by perimeter 806 to prevent entry of foreign substances, such as the ingress of surgical fluids.

Contact cover 406 is mounted atop switch contact 512 with its conductive perimeter edge 806 physically contacting outer terminal 702. FIG. 8B shows contact cover 406 in an initial position in which the bottom surface 804 of contact cover 406 is spaced from inner terminal 704. FIG. 8C shows contact cover 406 in a depressed position. Snap dome contact cover 406 is depressed by applying a force in direction F1 which causes the center of the contact cover 406 to invert, typically with a snap, and assume a depressed position. When in the depressed position, contact cover's 406 bottom surface 804 contacts inner terminal 704, thus electrically connecting inner and outer terminals 704 and 702, respectively.

As the contact cover passes through the snap phase the surgeon receives tactile feedback which can be readily felt or heard by the surgeon, thus enhancing the surgeon's control over the activation of the electrosurgical instrument 10. When the force is removed from contact cover 406, the contact cover 406 resumes its initial position as shown in FIG. 8B, e.g., snapping back, to its initial position.

Dome shaped contact cover 406 may be any geometric shape such as hemi-spherical or hemi-cylindrical. A variety of embodiments of a snap dome switch are taught in U.S. Pat. No. 6,747,218 which is incorporated herein by reference.

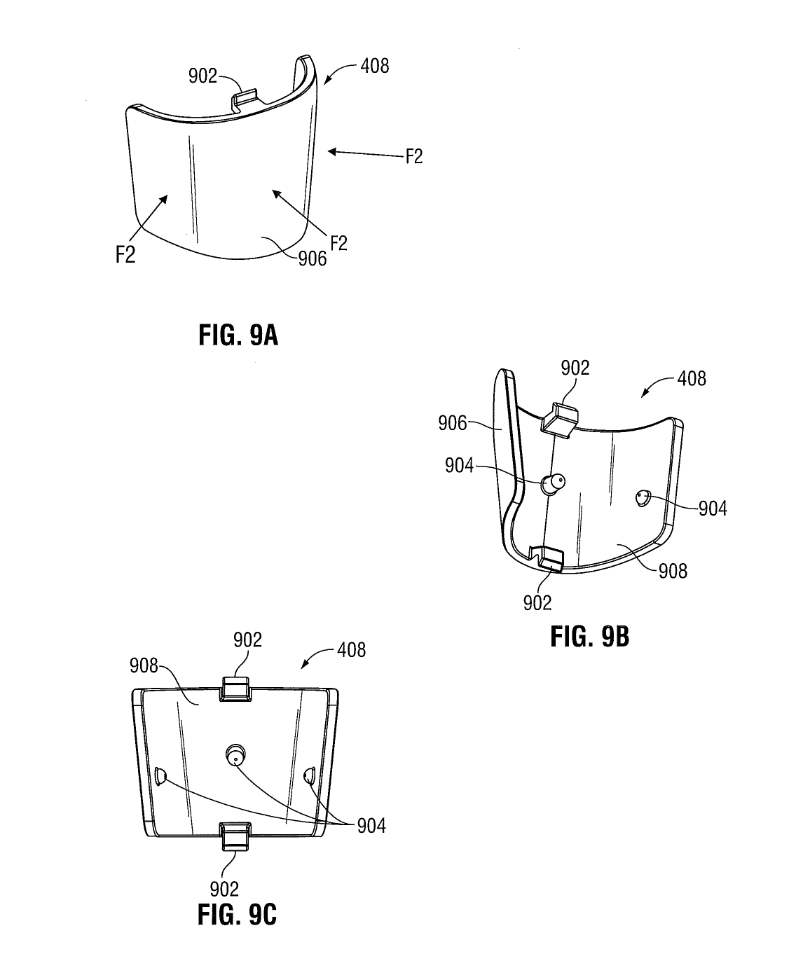

With reference to FIGS. 9A-9C, an exemplary wraparound cap 408 is shown. Cap 408 configured to mechanically engage the housing 20, e.g., by a snap fit, and positioned over an exposed portion of flexible circuit board 402, which includes switch contact segment 510. Exemplary snap fit flanges 902 engage the housing 20 for mounting the cap 408 to the housing 20. The method and structure for mounting cap 408 to the housing 20 is not limited to snap fit and flanges 902, and other methods and structures for mounting cap 408 to housing 20 are envisioned.

Cap 408's inner surface 908 is provided with a plurality of mechanical interfaces for interacting with switches 404a-404c. In the present example the mechanical interfaces include projecting structures, such as activation nubs 904, wherein each nub 904 corresponds to a control switch 404. When cap 408 is mounted to housing 20, each nub 904 is positioned opposite a corresponding control switch 404 so that application of a force in direction F2 to outer surface 906 of cap 408 and relative to the housing 20 causes a particular nub 904 to apply a force to the outer surface of the corresponding snap dome contact cover 406 associated with the corresponding control switch 404 along the X-X longitudinal axis for compressing the contact cover 406 and actuating the control switch 404. The force is typically applied to the outer surface 906 of the cap 408 by the surgeon's finger(s). In the configuration of the present example, the surgeon uses a thumb to apply pressure to cap 408.

Cap 408 may be formed of a rigid material, such as a hard plastic. As such, in order to activate the control switch 404 that corresponds to a selected nub, a force applied in direction F2 need not be applied to the outer surface 906 at the location of the selected nub 904, e.g., at a location on the outer surface 906 that is directly opposite the location of the selected nub on the inner surface 908. Provided that the force is applied in the vicinity of the selected nub 904, the rigidity of the cap 408 allows the force to be translated to the location of the nub for causing the nub 904 to compress the selected nub's snap dome contact cover 406. A single force is thus applied for activating a single control switch 404 that is located in the vicinity of the location where the force was applied to the cap 408. The snap of the snap dome contact cover 406 may be translated via the cap 408 to the surgeon's fingers so that the surgeon feels receives tactile feedback upon activation of the control switch 404.

Alternatively, cap 408 may be formed of a flexible, rubbery plastic. In this case, activation of a selected nub 904 would require applying a force at a location on the outer surface 906 that is substantially directly opposite the location of the selected nub 904 on the inner surface 908. In this embodiment, the surgeon may have the capability of selecting between control switches 404a-404c that are closely spaced and/or applying force at more than one location substantially simultaneously. The surgeon may have the capability of utilizing a variety of functions by selecting a particular control switch 404a-404c and/or combinations thereof to activate the electrosurgical instrument 10 (e.g., sequentially or simultaneously). The material forming cap 408 may be sufficiently thin so that the surgeon can feel the snap of the snap control contact cover 406 directly under his/her finger and through the cap 408.

While FIGS. 9A-9C show a one-to-one correspondence between the nubs 904 and the control switches 404a-404c, the disclosure is not limited thereto. Other embodiments are envisioned, including providing more than one closely spaced nub 904 to correspond to a single control switch 404a-404c for compressing its contact cover 406; or providing more control switches 404a-404c than nubs 904 in order that not every control switch 404a-404c may be activated by applying force to cap 408. This may be useful when different functions are assigned to different control switches 404a-404c as described below, and a particular model of the electrosurgical instrument 10 does not associate functionality with particular control switches 404a-404c. The cap 408 may be designed to only be capable of activating selected control switches 404a-404c. The nubs 904 of contact cover 406 may thus be designed to be compatible with the functionality provided by the processing device 1002 (see FIG. 10).

With reference to FIG. 10A, second electrical device 752 is shown, including a tangible processing device 1002 that receives a signal CS_{switch} which is indicative of actuation of one or more control switches 404a-404c. Processing device 1002 includes, for example, a microprocessor, a microcontroller, and/or a digital signal processor. The processing device 1002 may include a processor 1008, e.g., a central processing unit, and memory 1010, e.g., flash memory, RAM, or ROM. Internal to or accessible by processing device 1002 A control module 1012 including at least one series of programmable instructions may be stored in memory 1010 and executable by the processor 1008. In one embodiment, in addition to, or instead of processor 1008, memory 1010, and control module 1012, processing device 1002 may include a circuit, including analog, digital and/or logical devices, that processes the input and generates output.

There are a variety of ways in which CS_{switch} may be generated. Each switch 404a-404c outputs a signal S₄₀₄ₐ-S_{404c}, respectively, that indicates when the corresponding control switch 404a-404c is actuated. As shown in FIG. 10A, signals S₄₀₄ₐ-S_{404c} may all be tied to signal CS_{switch}, so that CS_{switch} indicates when any of the control switches 404a-404c have been actuated, without differentiating between which of the control switches 404a-404c was actuated. Processing device 1002 processes CS_{switch} and based on the processing results generates one or more control signals for controlling a device or circuit. In the current example, processing device 1002 generates CS1_{pd} and CS2_{pd}. CS1_{pd} controls generator 80 for controlling delivery of electrosurgical energy to the end effector assembly 100 for application of the electrosurgical energy to a patient. CS2_{pd} controls another electrical device 756, such as a knife driver for deploying or retracting knife 114. Additional circuitry may be provided, which may be integrated with processing device 1002 or physically separate from processing device 1002, that processes CS_{switch}, e.g., for recognizing the voltage level and/or recognizing rising and/or falling edges of CS_{switch}.

FIG. 10B illustrates another way in which CS_{switch} may be generated. Second electrical device 752 further includes a multiplexor (MUX) 1020. The signals S₄₀₄ₐ-S_{404c} output by switches 404a-404c, respectively, are received and processed by MUX 1020 for outputting CS_{switch}. CS_{switch} may indicate which of signals S₄₀₄ₐ-S_{404c}, were activated by actuation of a corresponding control switch 404a-404c.

Processing device 1002 may further include clock 1014 that may be used by processing device 1002 to determine the duration between actuations or of the actuations (e.g., how long the control switch 404a-404c is held in a depressed position for) of one or more of control switches 404a-404c. Processing device 1002 may process the determined duration and/or the sequence of actuations of control switches 404a-404c for controlling when to change operating modes or operating states. Additionally, the duration between actuations may be used to recognize the occurrence of multiple closely spaced actuations (e.g., the equivalent to a “double-click” or “triple-click”). Such multiple “clicks” may be used to provide additional functionality by assigning different functions to each different multiple click.

Control module 1012 includes an initiate seal operation (ISO) module 1016, and may further include a control seal operation (CSO) module 1018. ISO module 1016 controls initiation of a seal operation.

CSO module 1018 controls the generator 80, which may include processing data received by sensors (not shown), and outputting control signals CS1_{PD} to the generator 80 for controlling generation of the electrosurgical energy and adjusting parameters of the voltage and current output, such as magnitude and frequency. The CSO module 1018 may further control the output stage of the generator 80 for modulating the output electrosurgical energy, including adjusting waveform parameters, e.g., waveform shape, pulse width, duty cycle, crest factor, and/or repetition rate.

Additionally, the CSO 1018 may determine when a tissue seal operation is complete and/or successful, based upon feedback information provided by the generator 80 and/or sensors provided at the generator 80 or the surgical site. The determination may be based on one or more of the following parameters: tissue temperature, tissue impedance at the seal, change in impedance of the tissue over time and/or changes in the power or current applied to the tissue over time. Control of the sealing operation is described in U.S. patent application Ser. No. 12/246,553, which is incorporated herein by reference.

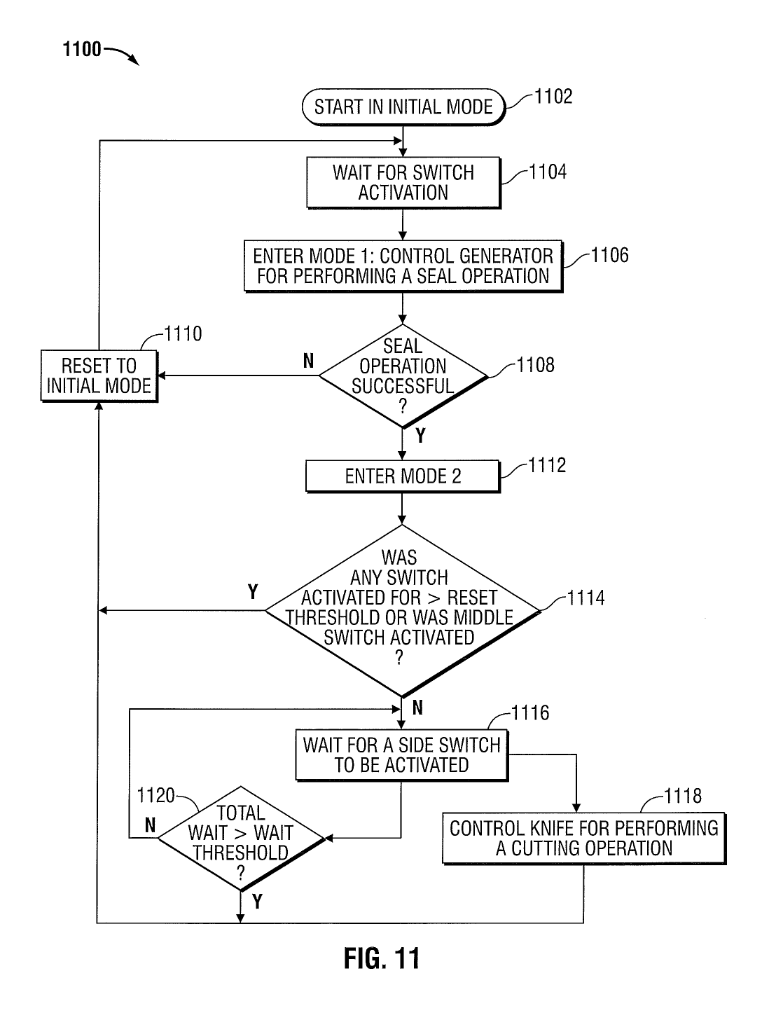

With reference to FIG. 11, flowchart 1100 shows a method executed by processing device 1002 which includes executing ISO module 1016 and CSO module 1018 for transitioning between exemplary modes of operation based on the sequence of actuations of control switches 404a-404c, the duration of time between actuations, and other conditions.

The method and a configuration using two side control switches 404a and 404c and one middle control switch 404b are provided as an example. Other methods and configurations are envisioned. For example, different sequences, combinations, or types of actuations (e.g., double click) of the control switches 404a-404c are envisioned. Such actuations may provide the same functionality described or provide different or additional functionality, e.g., control additional electrical devices 756. Furthermore, the control switches 404a-404c may be positioned differently, or fewer or more control switches 404a-404c may provided than the configuration shown or described.

At step 1102, operation of processing device 1002 begins in an Initial Mode. Once in the Initial Mode, the processing device 1002 continues to operate in the Initial Mode until ISO module 1016 determines that one or more of the control switches 404a-404c has been actuated. At wait step 1104, ISO module 1016 waits indefinitely for actuation of any of the control switches 404a-404c. ISO module 1016 detects actuation of any of the control switches 404a-404c at step 1104, without differentiating between which of control switches 404a-404c was activated. Accordingly, such detection has the same effect, regardless of which control switch 404a-404c is actuated and control proceeds to step 1106.

At step 1106, processing device 1002 enters Mode 1 in which ISO module 1016 commands CSO module 1018 to commence and control a seal operation. While in Mode 1, CSO module 1018 commences and controls the seal operation, such as by adjusting parameters of the voltage and current output by generator 80, e.g., modulating magnitude and frequency. Additionally, while in Mode 1, CSO module 1018 may further modulate the output electrosurgical energy, including by adjusting waveform parameters, e.g., waveform shape, pulse width, duty cycle, crest factor, and/or repetition rate.

In one embodiment, upon commencement of a seal operation, CSO module 1018 controls the seal operation until it is complete. In another embodiment, the ISO module 1016 instructs CSO module 1018 to perform the seal operation only for a duration of time that one or more of the control switches 404a-404c is actuated or until CSO module 1018 determines that the seal operation is complete, at which time a user indicator device (e.g., a visual or audio indicator (not shown)) is controlled to signal to the surgeon that the seal operation is complete. In this embodiment, the seal operation may be terminated before it is complete when the surgeon releases the actuating control switch 404a-404c.

Upon termination of the seal operation (either upon completion or by release of the actuating control switch 404a-404c), the CSO module 1018 outputs a signal indicating whether or not the seal operation was successful. A determination as to whether the seal operation was successful is typically based on feedback from one or more sensors sensing parameters, such as tissue temperature, tissue impedance at the seal, change in impedance of the tissue over time and/or changes in the power or current applied to the tissue over time. Once the seal operation is terminated and the CSO module 1018 has output the signal indicative of the seal operation's success, control passes to decision-step 1108.

At decision-step 1108, a determination is made whether the CSO module 1018 indicated that the seal operation was successful. If it was not, control passes to step 1110. At step 1110, processing device 1002 is reset to operate in the Initial Mode. After the reset operation is performed at step 1110, control passes to step 1104.

When the determination at decision-step 1108 is positive, control passes to step 1112. At step 1112, processing device 1002 enters Mode 2. When operating in Mode 2, ISO module 1016 differentiates between which of the control switches 404a-404c is actuated. At step 1114, a determination is made as to: a) whether any of the control switches 404a-404c (without differentiating between switches 404a-404c) is actuated (e.g., with force F1 applied to contact cover 406 so that contact cover 406 assumes a depressed position) and held in a depressed position for a time period that exceeds a predetermined reset threshold; or b) whether the middle control switch 404b is actuated. If the determination is positive, control passes to step 1110. If the determination is negative, control passes to step 1116.

At step 1116, ISO module 1016 waits for actuation of either side control switch 404a or 404c. Upon recognition of actuation of either side control switch 404a or 404c, control passes to step 1118. At step 1118, ISO module 1016 generates control signal CS2_{PD} which is used to control a drive mechanism included with electrical device 756 for deploying knife 114 to sever tissue for performing a cutting operation. Next, control passes to step 1110.

If neither side control switch 404a nor 404c was actuated, at predetermined timed intervals, control passes to step 1120. At step 1120, a determination is made if a total wait time (e.g., the sum of accumulated wait time during successive iterations of step 1116) spent waiting at step 1116 exceeds a predetermined wait threshold. If the determination is negative, control returns to step 1116. If the determination is positive, control passes to step 1110.

The disclosure is not limited to the design described in FIG. 11. Other sequences and/or combinations of actuations of control switches 404a-404c are envisioned for transitioning between a variety of operational modes. Regardless of whether one mode of operation is provided for, or the method governing the transition between modes of operation, actuation of control switches 404a-404c are performed by operating a single activator 40.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed:

1. An electrosurgical instrument, comprising:
a housing having a treatment portion attached at a distal end thereof and a handle for actuating the treatment portion;
a plurality of switches each configured to close an associated open circuit upon activation thereof for controlling respective functions or parameters associated with the treatment portion; and
a cap configured to mechanically engage the housing and having an inner surface including a corresponding number of mechanical interfaces configured to align with each switch such that an activation force against an outer surface of the cap and relative to the housing closes the open circuit associated with a corresponding switch.

2. The electrosurgical instrument according to claim 1, wherein:
at least one of the respective functions controlled by at least one of the switches is selected from the group consisting of coagulation, homeostasis, RF sealing, ultrasonic sealing, blending, fulguration, coagulation, cauterization, cutting, and stapling tissue,
at least one of the respective parameters controlled by at least one of the switches is selected from the group consisting of treatment mode, energy signal amplitude, energy signal frequency, energy signal waveform shape, energy signal duty cycle, and energy signal root mean square, and
the electrical signal is selected from the group of energy signals consisting of electrical current, electrical voltage, electrical power, and ultrasound signal.

3. The electrosurgical instrument according to claim 1, wherein the plurality of switches are disposed on a printed circuit board that is coupled to an external surface of the housing.

4. The electrosurgical instrument according to claim 1, wherein:
the housing has a longitudinal axis, when a first switch of the plurality of switches is depressed in a direction along the longitudinal axis of the housing by a corresponding one of the mechanical interfaces, a first function or parameter of the respective functions or parameter associated with the treatment portion is controlled, and
when a second switch of the plurality of the switches is depressed in a direction along the longitudinal axis of the housing by a corresponding one of the mechanical interfaces, a second function or parameter of the respective functions or parameters associated with the treatment portion is controlled.

5. The electrosurgical instrument according to claim 1, wherein the cap is ergonomically configured to wrap around the housing.

6. The electrosurgical instrument according to claim 1, wherein:
the plurality of switches are configured as dome switches having a first and second terminal and a cover with a conductive inner surface, and
the open circuit associated with a corresponding switch is closed when the corresponding mechanical interface applies a second force to an outer surface of the cover causing the cover to move from an initial first position in which the inner surface of the cover does not electrically conduct between the first and second terminals to a second position in which the inner surface does electrically conduct between the first and second terminals.

7. The electrosurgical instrument according to claim 1, further comprising:
at least one tangible processing device; and
a module including a plurality of programmable instructions executable by the at least one processing device for:
receiving at least one control signal indicative of actuation of plurality of switches of the at least one switch; and
controlling the respective functions or parameters based on at least one of which switch of the plurality of switches is actuated, and timing between successive actuations, as indicated by the received at least one control signal.

8. The electrosurgical instrument according to claim 7, wherein:
the module operates in a first and second mode, when operating in the first mode, when a switch of the plurality of switches is actuated the module controls a first function or parameter of the parameters or functions, and
when operating in the second mode, when the switch is actuated the module controls a second function or parameter of the parameters or functions.

9. The electrosurgical instrument according to claim 8, further comprising an electrosurgical generator and a knife driver configured to operate a knife for performing a cutting operation, wherein:
the first function includes controlling the electrosurgical generator to perform a sealing operation that includes generating electrosurgical energy for performing a seal operation to seal tissue, and
the second function includes controlling the knife driver to perform a cutting operation to sever tissue.

10. The electrosurgical instrument according to claim 8, wherein:
the module begins operating in an initial resting mode, the module transitions from operating in the initial resting mode to operating in the first mode upon actuation of the plurality of switches in a first predetermined fashion, the module transitions from operating in the first mode to operating in the second mode upon completion of a successful seal operation, and
the module controls terminating an active sealing operation and transitions from operating in the first or second mode to operating in the initial resting mode upon actuation of the plurality of switches in a second predetermined fashion different from the first predetermined fashion.

11. An electrosurgical instrument, comprising:
a housing having a treatment portion attached at a distal end thereof and a handle for actuating the treatment portion;
at least one switch each configured to close an associated open circuit upon activation thereof for controlling at least one respective function or parameter associated with the treatment portion; and
wherein the at least one switch is disposed on a printed circuit board that is coupled to an external surface of the housing.

12. The electrosurgical instrument according to claim 11, wherein:
the at least one switch is configured as a dome switch having a first and second terminal and a cover with a conductive inner surface, and
the open circuit associated with a corresponding switch of the at least one switch is closed when a force is applied to an outer surface of the cover causing the cover to move from an initial first position in which the inner surface of the cover does not electrically conduct between the first and second terminals to a second position in which the inner surface does electrically conduct between the first and second terminals.

13. The electro surgical instrument according to claim 12, further comprising a single cap having an inner and outer surface configured to couple to the housing so that the circuit board is disposed in between the cap and the housing with its outer surface exposed and its inner surface having a number of mechanical interfaces configured to align with each switch of the at least one switch such that application of an activation force against an the outer surface of the cap and relative to the housing closes the open circuit associated with a corresponding switch of the at least one switch.

14. The electrosurgical instrument according to claim 11, further comprising:
at least one tangible processing device; and
at least one software module including a plurality of programmable instructions executable by the at least one processing device for:
receiving at least one control signal indicative of actuation of two or more switches of the at least one switch; and
controlling the at least one respective function or parameter based on at least one of which switch of the at least one switch is actuated, and timing between successive actuations, as indicated by the received at least one control signal.

15. An electrosurgical instrument, comprising:
a housing having a treatment portion attached at a distal end thereof and a handle for actuating the treatment portion;
an electromechanical interface for coupling to an electrosurgical energy generator that generates electrosurgical energy which is provided to the treatment portion; and
at least one switch disposed on a printed circuit board that is coupled to an external surface of the housing and configured to close an associated open circuit upon activation thereof for controlling operation of the electrosurgical generator.

16. The electrosurgical instrument according to claim 15, further comprising a single cap configured to mechanically engage the housing so that the circuit board is disposed in between the cap and the housing so that the cap's outer surface is exposed and its inner surface faces the plurality of switches, such that an activation force against an outer surface of the cap and relative to the housing closes the open circuit associated with a corresponding switch of the at least one switch.

17. The electrosurgical instrument according to claim 15, wherein closing the associated open circuit further controls at least one additional function or parameter associated with the treatment portion, the electrosurgical instrument further comprising:
at least one tangible processing device;
at least one software module including a plurality of programmable instructions executable by the at least one processing device for:
receiving at least one control signal indicative of actuation of one or more switches of the plurality of switches; and
controlling the electrosurgical generator and the at least one additional function or parameter based on at least one of which switch of the plurality of switches is actuated, and timing between successive actuations as indicated by the received at least one control signal.
